(19) United States
(12) Patent Application Publication
(10) Pub. No.: US 2024/0127932 A1
(43) Pub. Date: Apr. 18, 2024

(54) Healthcare Provider Directory

(71) Applicant: Electronic Referral Manager Inc., Mt. Pleasant, SC (US)

(72) Inventors: David Bongiovanni, Mt. Pleasant, SC (US); Ivan Nolasco, Santo Domingo (DO); Richard Hammer, Tucson, AZ (US)

(73) Assignee: Electronic Referral Manager Inc., Mt. Pleasant, SC (US)

(21) Appl. No.: 17/964,182

(22) Filed: Oct. 12, 2022

(51) Int. Cl. G16H 40/20; G06F 16/22; G06F 16/242

(52) U.S. Cl. CPC G16H 40/20; G06F 16/2282; G06F 16/244

(57) ABSTRACT

A method comprising receiving referral data and National Provider Identifier (NPI) data, creating a database and tables that include a practitioner table, an organization table, a join table, and an intermediary table, importing the referral data and the NPI data into the intermediary table, processing an NPI practitioner record, processing an NPI organizational record, joining the practitioner record to the organization table, processing referral data, and presenting a database search interface to a user.

### BACKGROUND

The present invention relates to managing healthcare referrals, and more specifically, to searching for healthcare providers when sending referrals and scheduling appointments.

In the medical industry, patients are referred by one healthcare provider to another healthcare provider. Often a first healthcare provider may diagnose a patient with a medical condition that should be treated by a specialist second healthcare provider. For example, a primary care physician may care for a patient with an ankle injury. The primary physician may believe that the patient should see an orthopedic specialist and decide to refer the patient to a particular orthopedic specialist. An appointment may be scheduled with the orthopedic specialist to evaluate and care for the ankle of the patient.

### SUMMARY

Embodiments of the present invention are directed to a method comprising receiving referral data and National Provider Identifier (NPI) data, creating a database and tables that include a practitioner table, an organization table, a join table, and an intermediary table, importing the referral data and the NPI data into the intermediary table, processing an NPI practitioner record, processing an NPI organizational record, joining the practitioner record to the organization table, processing referral data, and presenting a database search interface to a user.

Embodiments of the present invention are directed to a system comprising a processor operative to receive referral data and National Provider Identifier (NPI) data, create a database and tables that include a practitioner table, an organization table, a join table, and an intermediary table, import the referral data and the NPI data into the intermediary table, process an NPI practitioner record, process an NPI organizational record, join the practitioner record to the organization table, process referral data, and a display operative to present a database search interface to a user.

Embodiments of the present invention are directed to a non-transitory computer readable medium including the instructions of receiving referral data and National Provider Identifier (NPI) data, creating a database and tables that include a practitioner table, an organization table, a join table, and an intermediary table, importing the referral data and the NPI data into the intermediary table, processing an NPI practitioner record, processing an NPI organizational record, joining the practitioner record to the organization table, processing referral data, and presenting a database search interface to a user.

### BRIEF DESCRIPTION OF THE DRAWINGS

FIG. 1 illustrates a block diagram of an example computer system.

FIG. 2 illustrates a block diagram of an exemplary method of processing and presenting referral and NPI data as part of the referral and appointment process.

FIG. 3 illustrates a block diagram of an exemplary method for importing NPI data into an intermediary table.

FIG. 4 illustrates a block diagram of an exemplary method for importing referral data into an intermediary table.

FIG. 5 illustrates a block diagram of an exemplary method for processing deactivation records.

FIG. 6 illustrates an exemplary method for processing practitioner records.

FIG. 7 illustrates a block diagram of a method of processing NPI organizational records.

FIG. 8 illustrates a block diagram of a method of joining practitioner records to organization records.

FIG. 9 illustrates a block diagram of a method for processing the referral data.

FIG. 10 illustrates a block diagram of an exemplary method for exporting searchable fields to memory data stores.

FIG. 11 illustrates an example of a user interface for searching a database.

### DETAILED DESCRIPTION

Although this disclosure includes a detailed description of a computing environment, the teachings herein are not limited to the described computing environment. In embodiments, any implementation of a computer environment may be used whether now understood or later developed.

As discussed above, the healthcare referral process often involves a first healthcare provider evaluating a patient and deciding to refer the patient to another healthcare provider (a receiving provider) who will continue to evaluate and care for the patient. Such a referral process allows patients to receive the care they need often from medical or other subject matter experts.

One challenge for referring patients to other medical professionals is finding the correct healthcare providers for the patients. In this regard, there are many considerations that should be addressed when making a referral. The referring provider should identify a receiving healthcare provider who practices a desired type of medicine. Other considerations include, for example, convenience or practicality of the geographic location of the receiving provider with respect to the patient, whether the receiving provider accepts new patients, and the gender of the provider. Other considerations include languages spoken by the provider and the type of medical insurance accepted by the provider among others.

Another feature of the system includes marketing an outreach so that a user may find providers by searching. The results of these searches may be used for marketing purposes.

In this regard, a system that may access data associated with referring patients to a receiving provider and provide a user interface to facilitate accessing this data in a usable format is desired. The data used to find a receiving provider may be found from a variety of sources. For example, one data source may be the Centers for Medicare and Medicaid Services (CMS) referral data. Another source for data may include CMS National Provider Identifier (NPI) data.

In embodiments described herein, a method for developing a system for identifying and facilitating referrals is provided. The system includes an aggregation of data used for referrals in a useful format and a user interface that allows users to access the aggregated data and make decisions regarding referring patients to a referring provider.

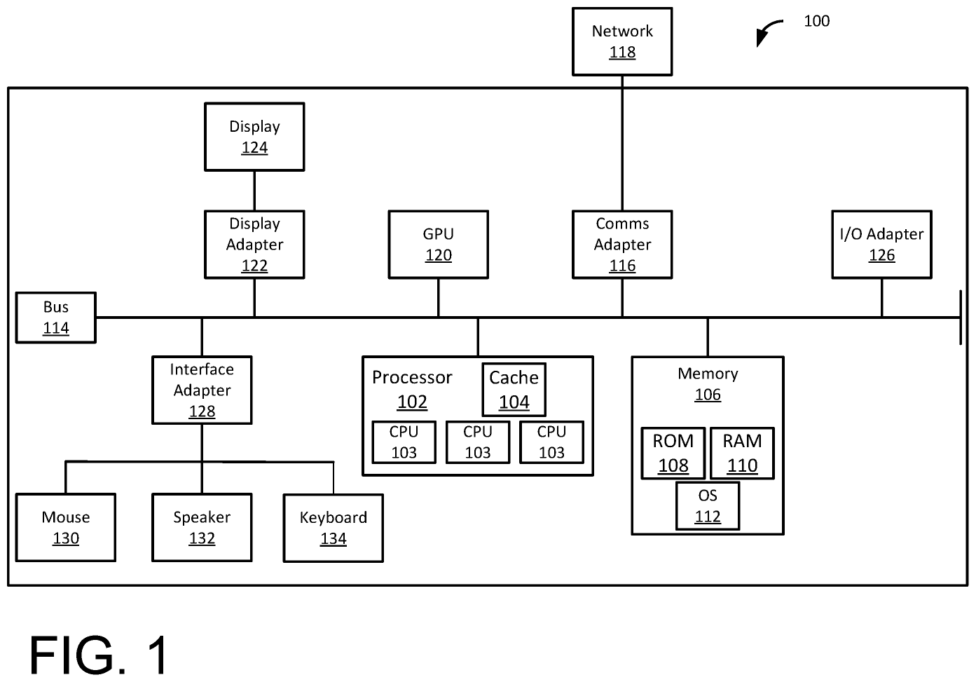

FIG. 1 illustrates a block diagram of an example computer system 100. In embodiments, a microprocessor is arranged in a personal computer, workstation, minicomputer, or mainframe computer. Embodiments system 100 a mobile device or part of a mobile device.

The computer 100 includes processor 102 communicatively connected to an input/output (I/O) adapter 126, memory 106, a communications adapter 116, an interface adapter 128, a display adapter 122, and a graphics processing unit (GPU) 120 via a system bus 114.

The processor 102 includes any number of central processing units (CPU) 103 and a cache memory 104. The CPU 102 is operable to perform any number of processing tasks by executing program instructions. In embodiments the processor 102 may include any number of suitable auxiliary processors or microprocessors. The processor 102 may include a cache memory 104 that is often operative to increase the speed of processing tasks performed by the processor 102.

The I/O adapter 126 connects a variety of input and output devices to the computer 100. The memory 106 may include any type of suitable memory such as, for example, magnetic, solid state, disk-based storage, drum-based storage, random access memory (RAM) 110, and read only memory (ROM) 108. The operating system of the system 100 may be stored in the memory 106. The communications adapter 116 is operative to facilitate communications between the system 100 and a network 118. The network 118 may include, for example, a local area network, a wide area network or the Internet. The system 100 may connect to the network 118 via, for example, a wired or wireless connection. The display adapter 122 is operative to communicably connect a display 124 to the system 100. The display may include any type of suitable display such as, for example, a desktop display, a touch screen, or a mobile display. The GPU 120 includes a processor that is operative to generate graphical data that may be sent to the display 124 for presentation to a user. The interface adapter 128 provides an interface between peripheral devices and the system bus 114. The peripheral devices may include, for example, a mouse 130, a speaker 132, and a keyboard 134, sensors, and actuators, among others.

The memory 106 may store computer-readable and computer-executable instructions. The instructions implement logical functions. The operating system 112 may control the operations of the system 100 and the functions of other software programs or operations.

Memory can also include program instructions for Engine 1, configured to improve the wellness of a user.

According to some embodiments, computer 100 can include a mobile communications adapter 123. Mobile communications adapter 123 can include GPS, cellular, mobile, and/or other communications protocols for wireless communication.

In some embodiments, computer 100 can further include communications adapter 116 for coupling to a network 118.

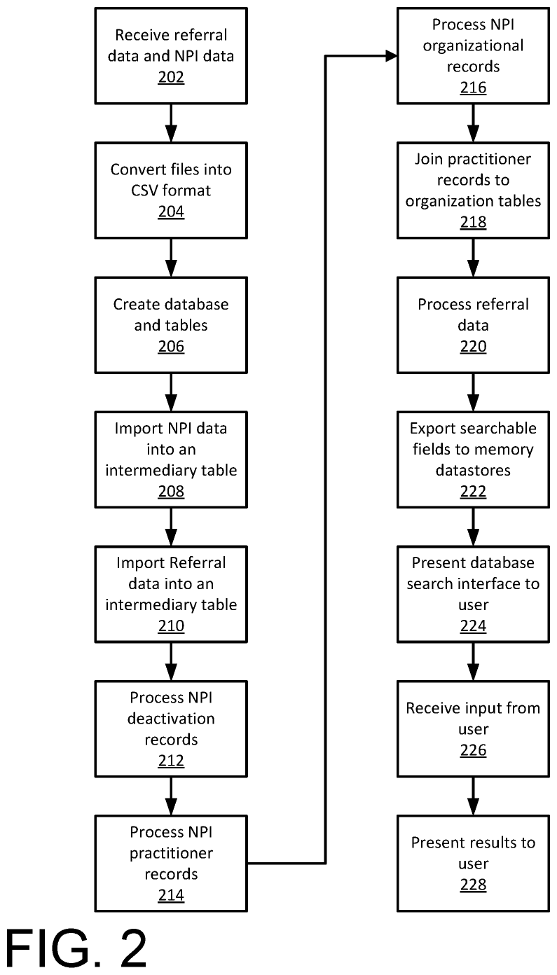

FIG. 2 illustrates a block diagram of an exemplary method of generating and operating a referral system that is operative to facilitate a user in making patent referrals.

The referral system is operative to use data to select providers for referrals. The system may create a list of providers using data described below. For example, the user may retrieve a list of doctors that are within a particular geographic location. In another example the list of providers may be ranked according to the criteria selected by the user.

In block 202 the system 100 (of FIG. 1) receives data. The data received includes, for example, referral data from the Centers for Medicare and Medicaid Services (CMS) referral data from CMS may include for example National Provider Identifier data of a provider, which providers are involved in a referral from one provider to another, and current procedural technology (CPT) codes. Referral data may also include an IDC-10 code and other medical or demographic details about specific referrals. National Provider Identifier (NPI) data may include, for example, an NPI number, name, organization name and address. Data sources are not limited to these examples and may include data from any appropriate sources.

The referral data may include any number of types of data. For example, the referral data may include, a doctor (provider) database that may be retrieved from the NPI data. The doctor database includes the name of a provider, the specialty of a provider, a hospital associated with the provider, who the provider sends referrals to and who the provider receives referrals from. Other data includes whether the provider will accept new patients, the insurance network accepted by the provider, the specialties of the provider, common procedures performed by the provider, demographic information, phone numbers and fax numbers, average wait and response times, the language spoken by the provider, gender, International Classification of Diseases, 10^{th} revision (ICD-10), and the geographic location of the provider. The IDC-10 is a classification and coding system for medical diagnoses and procedures managed by the World Health Organization. The doctor database may also include, for example, average wait and response times, healthcare provider ratings and reviews, fees for services, associations with groups, education, specialties, titles, and credentials. Other categories that may be included include Current Procedural Terminology (CPT) codes which is a classification and coding system for medical procedures and services managed by the American Medical Association. Taxonomy codes may be included; taxonomy codes are a classification and coding system for healthcare provider specialties managed by the National Uniform Claim Committee (NUCC). Other codes may include, for example, Systemized Nomenclature of Medicine Clinical Terms classification and coding system for medical terms. This may include conditions and procedures and is managed by the International Health Terminology Standards Development Organization (IHTSDO).

In block 204, the data from each data source is converted into coma-separated value (CSV) format. Coma separated value format is a plain text file that includes lists of data separated by comas.

In block 206, a database is created that includes tables. The tables include intermediary tables, practitioner tables, organization tables, and referral tables. The intermediary table is a table used to temporarily store data imported from a CSV file the intermediary table may include a single or multiple tables. The practitioner table includes multiple tables joined by foreign keys to store practitioner data. Practitioner tables include NPI data from healthcare practitioners or healthcare providers (providers). The practitioner data includes, for example, provider offices, demographic data about the providers, provider medical or service specialties, and geographic location data.

The organization tables include multiple tables joined by foreign keys to store organization data. An organization may include, for example, an office of a practitioner, a healthcare group, or a hospital. The organization tables include organization data that includes, for example, demographics about the organization, locations of the organization, addresses, phone numbers, fax numbers, and practice or service specialties of the organization. Practice specialties include medical services provided by the organization.

Referral tables include multiple tables jointed by foreign keys to store referral data. The join tables are tables split into multiple tables that include provider data with foreign key relationships to keep relationships between the data.

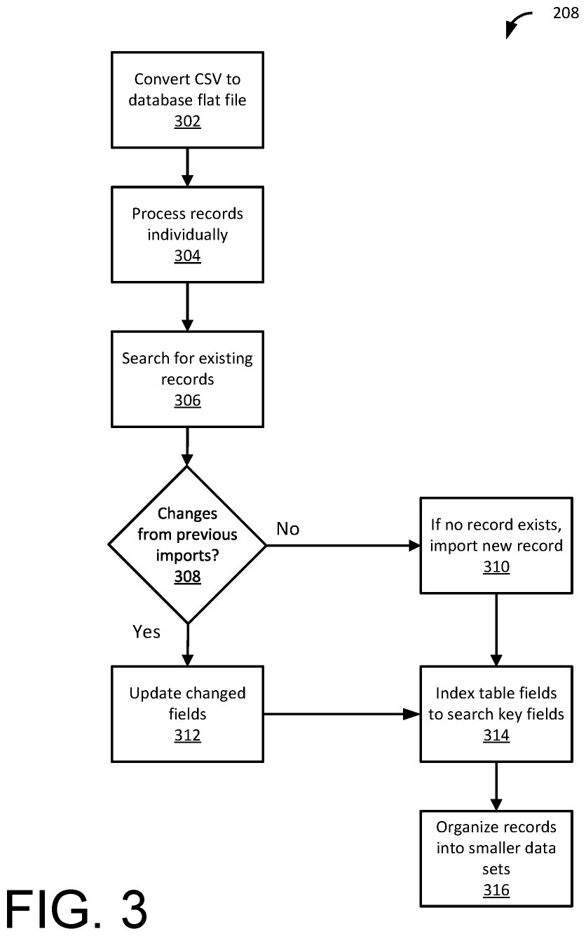

In block 208 the NPI data is imported into an intermediary table. FIG. 3 illustrates a block diagram of an exemplary method for importing NPI data into an intermediary table. The intermediary table is a table that is used store data like the CSV data. Database queries may be run using the data in the intermediate table to search and sort the data to convert the data to a relational database structure. This makes the data easily searchable. In this regard, in block 302, the CSV NPI data is converted into a database flat file. The records may be processed individually in block 304. In block 306, the system 100 searches for existing records in the database. The existing records are records in the database that are also in a second database that includes similar records. The system 100 determines whether there are changes from previous imports in block 308. If no record exists, new record is imported in block 310. If there are changed from previous imports in block 308, changed fields are updated in block 312. In block 314, the table fields are indexed to search for key fields. In block 316, the records are organized into smaller data sets to facilitate later processing. The smaller data sets are used to process the data efficiently.

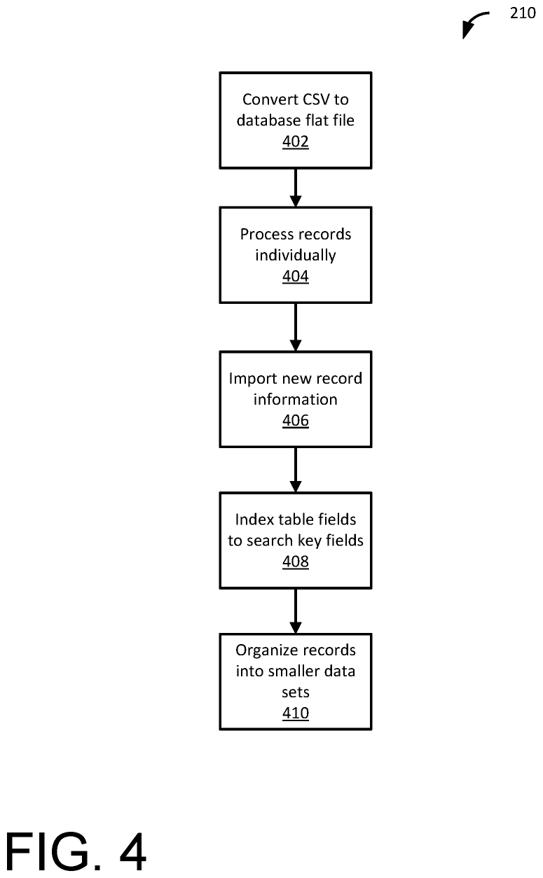

Referring to FIG. 2, in block 210 referral data is imported into an intermediary table. FIG. 4 illustrates a block diagram of an exemplary method for importing referral data into an intermediary table. Referral data includes anonymized records of patent referrals from Medicare data and other sources published by the government and other entities. In this regard, the CSV referral data is converted into a database flat file in block 402. In block 404, records are processed individually to format and categorize the data. New record information is imported in block 406. In block 408, table fields are indexed to search on key fields for processing described below. In block 410, the records are organized into smaller data sets or relational tables to facilitate later processing. The smaller data sets are used to process the data efficiently.

Referring to FIG. 2, in block 212 NPI deactivation records are processed. Deactivation records may include, for example, a practitioner who is no longer active such as a doctor who has stopped practicing medicine. Other deactivation records may include an organization that has ceased to function.

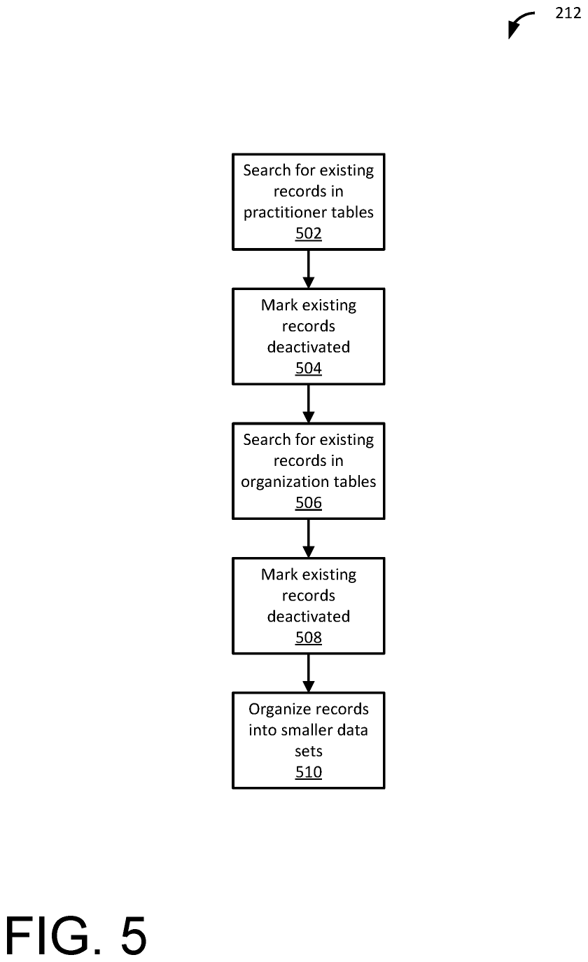

FIG. 5 illustrates a block diagram of an exemplary method for processing deactivation records. In block 502, existing records are searched for in the practitioner tables. Existing records include records for healthcare providers who have been imported into a second (target) database. In block 504, existing records are marked deactivated. In block 506, existing records are searched for in the organization records. The existing records are marked as deactivated in block 508. In block 510, the records are organized into smaller data sets to facilitate later processing. The smaller data sets are used to process the data efficiently.

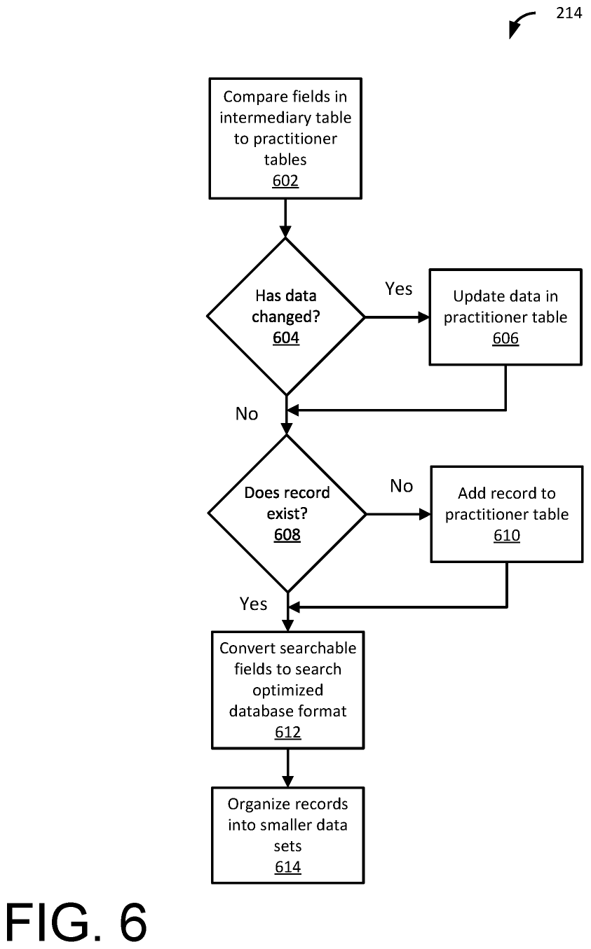

Referring to FIG. 2, in block 214 NPI practitioner records are processed. In this regard, FIG. 6 illustrates an exemplary method for processing practitioner records. Block 602 includes comparing fields in the intermediary table to the practitioner tables. In block 604 the system 100 determines if the data has changed. If the data has changed, the data in the practitioner table is updated in block 606. If the data has not changed, the system 100 determines if a record exists in block 608. If the record does not exist, the record is added to the practitioner table in block 610.

The searchable fields are converted to a search optimized database format in block 612. A search optimized database format may be generated in a number of steps. Some or all of the steps include, for example, formatting data for partial string searches, formatting data for case variations, formatting data for special character searching, converting data to be compatible with search optimized databases such as, for example, Elastic Search or GraphDB, and formatting data to match pre-determined lists of data such as city and state. Other steps may include, for example, formatting phone numbers, zip codes, and other non-textual data, and formatting data to limit common words, short words, or other character sequences which may confuse search results. The In block 614, the records are organized into smaller data sets to facilitate later processing. The smaller data sets are used to process the data efficiently.

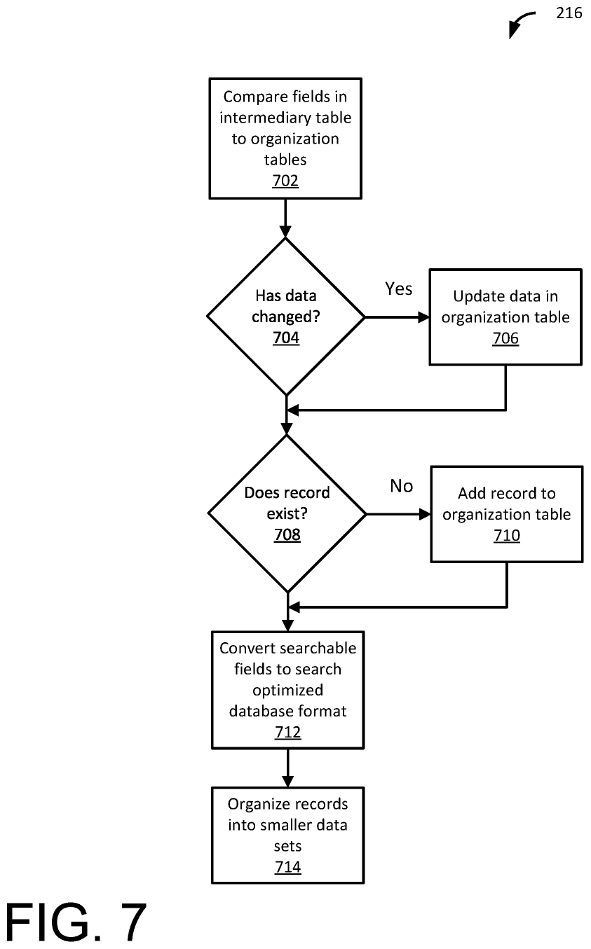

Referring to FIG. 2, in block 216, the system processes NPI organizational records. FIG. 7 illustrates a block diagram of a method of processing NPI organizational records. In this regard, in block 702 the fields in the intermediary table are compared to the fields in the organization tables. In block 704, the system determines if the data has changed. If yes, the data in the organization table is updated in block 706. If no, the system determines whether a record exists in block 708. If no, in block 710, the appropriate record is added to the organization table. In block 712, the organization table is converted to include searchable fields in an optimized database format. In block 714, records are organized into smaller data sets. The smaller data sets are used to process the data efficiently.

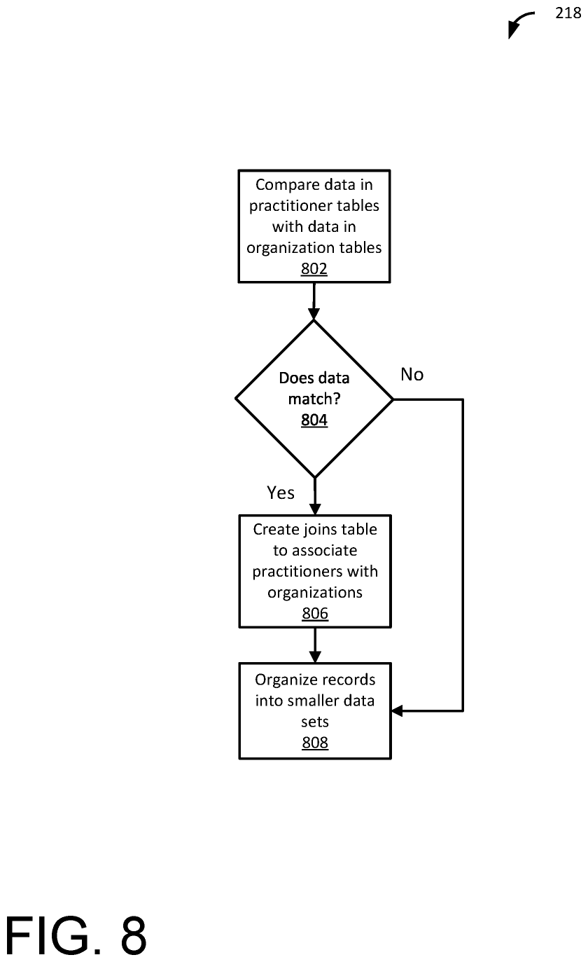

Referring to FIG. 2, in block 218 practitioner records are joined to organization records. FIG. 8 illustrates a block diagram of a method of joining practitioner records to organization records. In this regard, in block 802, data in the practitioner tables is compared with data in the organization tables. The data in the practitioners data is compared with the organization tables such that each practitioner is matched with one or more organizations and each organization is matched with all practitioners. In 804 the system determines whether the data in the practitioner tables and the data in the organization tables matches. If yes, a joins table is created to associate practitioners with organizations in block 806. In block 808, records are organized into smaller data sets. The smaller data sets are used to more efficiently process the data.

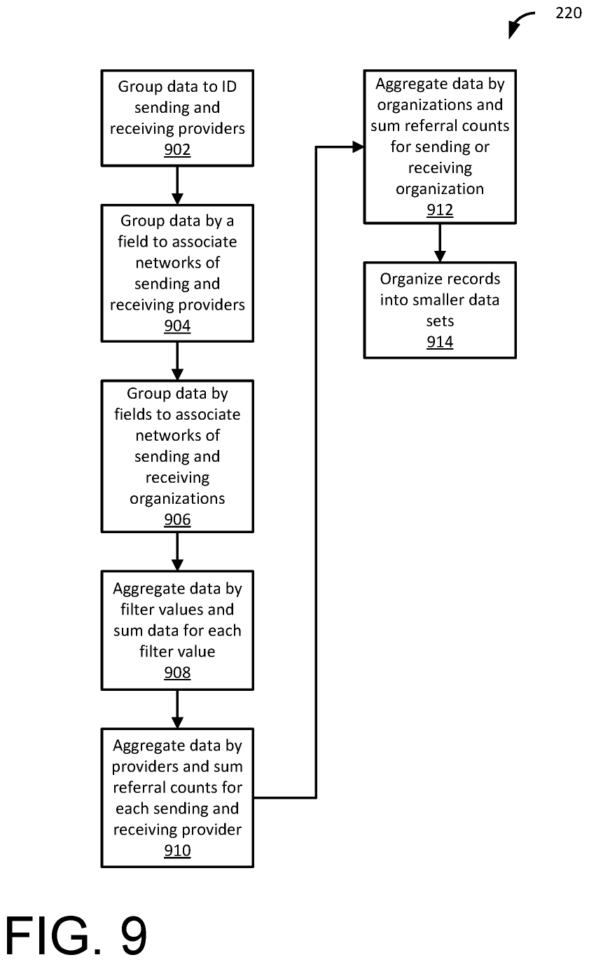

Referring to FIG. 2, in block 220 the system processes the referral data. FIG. 9 illustrates a block diagram of a method for processing the referral data. In this regard, in block 902, data is grouped to identify sending and receiving providers. In block 904, data is grouped by fields to associate networks of sending and receiving partners. Data is grouped by fields to associate networks of sending and receiving organizations in block 906. In block 908, data is aggregated by filter values and the data is summed for each filter value. A filter value may be used to classify data by any number of several parameters, for example, healthcare provider, organization, geographic region, condition, time frame, and patient demographic data among others. In block 910, the system aggregates data by providers and referral counts are summed for each sending and receiving provider. Referral counts include a tabulated total of referrals sent and received for a practitioner or organization along with one or more of the filter values. In block 912, data is aggregated by organizations and referral counts are summed for sending and receiving organizations. In block 912, records are organized into smaller data sets. Data sets are organized into smaller data sets to process the data more efficiently.

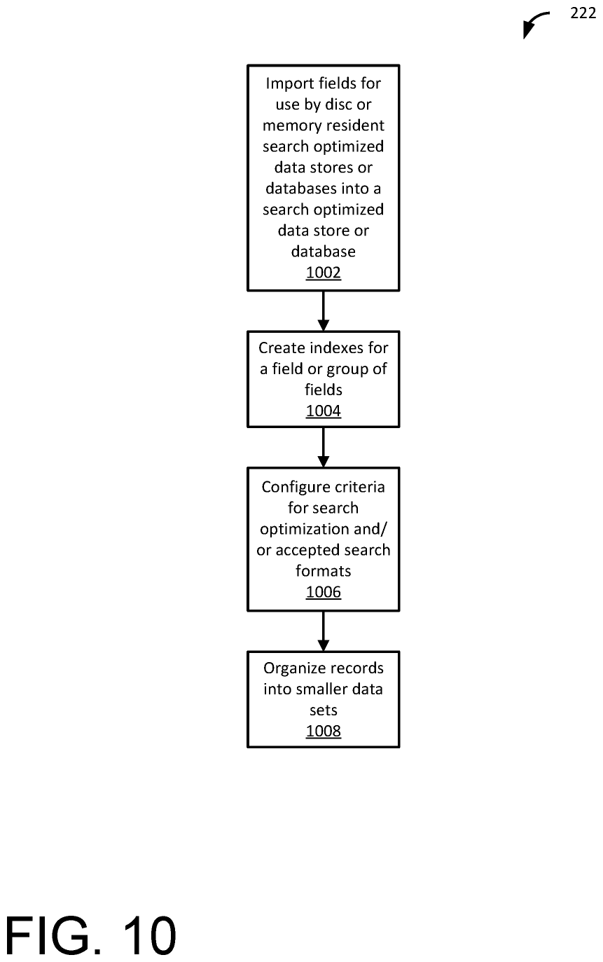

Referring to FIG. 2, in block 222, searchable fields are exported to memory data stores. FIG. 10 illustrates a block diagram of an exemplary method for exporting searchable fields to memory data stores. In block 1002, fields are imported for use by disc or memory resident search optimized data stores or databases into a search optimized data store or database. In block 1004, indexes for a field or groups of fields are created. The criteria for search optimization and/or accepted search formats are configured in block 1006. In block 1008, the records are organized into smaller data sets. The smaller data sets are used to process the data more efficiently.

Referring to FIG. 2, in block 224 the database search interface is presented to a user. In block 226, an input associated with a search is received from a user and input into the system. In block 228, the results of the search are presented to a user via an output device such as, for example, a display.

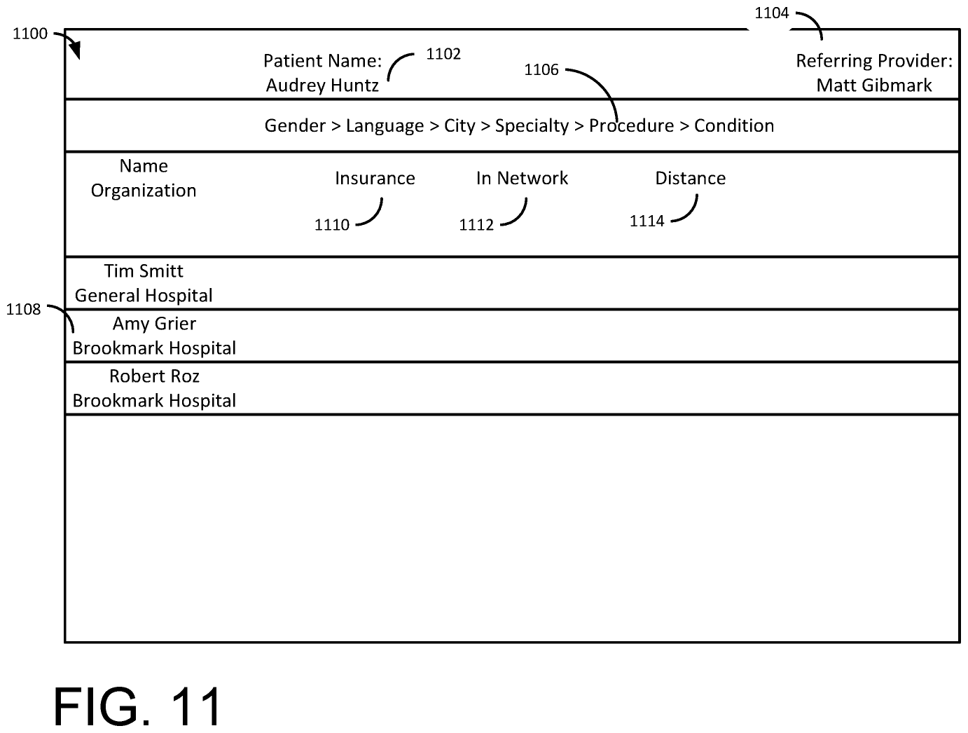

FIG. 11 illustrates an example of a user interface 1100 for searching the database described herein. The user interface 1100 is merely an example, any suitable user interface may be used. In this regard, the patient name 1102 and the referring provider 1104 are arranged in the user interface 1100. When a user conducts a search they may use search criteria 1106 to modify their search. Search criteria may include, for example gender of a provider, the language spoken by the provider, the geographic location of the provider, the practice specialty of a provider, the medial conditions treated by the provider, the hospital associated with a provider, whether the provider is accepting new patients, demographic information about a provider, the contact information associated with the provider such as, for example, phone numbers, fax numbers, and web addresses of the provider. Other information may include, for example distance from the user to the provider.

The list of providers 1108 which may, for example, include the name of a provider and their affiliated organizations is shown in FIG. 11. The list of providers may include an insurance column 1110 that includes insurance honored by the provider. A network column 1112 that includes the network of the provider, and a distance column 1114 that includes a distance from the user to the provider.

The embodiments described herein provide a system and method for providing searchable data for a user. The searchable data includes healthcare providers information to allow a user to search for healthcare providers and facilitate a medical referral process.

What is claimed is:

1. A method comprising:
receiving referral data and National Provider Identifier (NPI) data;
creating a database and tables that include a practitioner table, an organization table, a join table, and an intermediary table;
importing the referral data and the NPI data into the intermediary table;
processing an NPI practitioner record;
processing an NPI organizational record;
joining the practitioner record to the organization table;
processing referral data; and
presenting a database search interface to a user.

2. The method of claim 1, further comprising:
following the importing the referral data and the NPI data into the intermediary table, processing an NPI deactivation record.

3. The method of claim 1, wherein the processing the NPI practitioner records includes converting a searchable field into a search optimized database format.

4. The method of claim 1, wherein the processing the NPI organizational record includes converting a searchable field into a search optimized database format.

5. The method of claim 1, wherein the joining practitioner records to the organization table includes:
comparing data in the practitioner table with data in the organization table such that each practitioner is matched with an organization and each organization is matched with all of the practitioners;
determining whether the data in the practitioner table matches the data in the organization table; and
creating a joins table that associates a practitioner with an organization.

6. The method of claim 1, wherein the processing referral data includes:
grouping the referral data to identify sending and receiving providers;
grouping data by a filed to associate networks of sending and receiving providers;
grouping data by fields to associate networks of sending and receiving organization;
aggregating data by filter values and sum data for each filter value;
aggregating data by providers and summing referral counts for each sending and receiving provider; and
aggregating data by organizations and sum referral counts for sending and receiving organization.

7. The method of claim 1, wherein the referral data includes Centers for Medicare and Medicaid Services (CMS) referral data.

8. A system comprising:
a processor operative to:
receive referral data and National Provider Identifier (NPI) data;
create a database and tables that include a practitioner table, an organization table, a join table, and an intermediary table;
import the referral data and the NPI data into the intermediary table;
process an NPI practitioner record;
process an NPI organizational record;
join the practitioner record to the organization table;
process referral data; and
a display operative to present a database search interface to a user.

9. The system of claim 8, wherein the processor is further operative to:
following the importing the referral data and the NPI data into the intermediary table, processing an NPI deactivation record.

10. The system of claim 8, wherein the processing the NPI practitioner records includes converting a searchable field into a search optimized database format.

11. The system of claim 8, wherein the processing the NPI organizational record includes converting a searchable field into a search optimized database format.

12. The system of claim 8, wherein the joining practitioner records to the organization table includes:
comparing data in the practitioner table with data in the organization table;
determining whether the data in the practitioner table matches the data in the organization table; and
creating a joins table that associates a practitioner with an organization.

13. The system of claim 8, wherein the processing referral data includes:
grouping the referral data to identify sending and receiving providers;
grouping data by a filed to associate networks of sending and receiving providers;
grouping data by fields to associate networks of sending and receiving organization;
aggregating data by filter values and sum data for each filter value;
aggregating data by providers and summing referral counts for each sending and receiving provider; and
aggregating data by organizations and sum referral counts for sending and receiving organization.

14. The system of claim 8, wherein Centers for Medicare and Medicaid Services (CMS) referral data.

15. A non-transitory computer readable medium including the instructions of:
receiving referral data and National Provider Identifier (NPI) data;
creating a database and tables that include a practitioner table, an organization table, a join table, and an intermediary table;
importing the referral data and the NPI data into the intermediary table;
processing an NPI practitioner record;
processing an NPI organizational record;
joining the practitioner record to the organization table;
processing referral data; and
presenting a database search interface to a user.

16. The computer readable medium of claim 15, further comprising:
following the importing the referral data and the NPI data into the intermediary table, processing an NPI deactivation record.

17. The computer readable medium of claim 15, wherein the processing the NPI practitioner records includes converting a searchable field into a search optimized database format.

18. The computer readable medium of claim 15, wherein the processing the NPI organizational record includes converting a searchable field into a search optimized database format.

19. The computer readable medium of claim 15, wherein the joining practitioner records to the organization table includes:
comparing data in the practitioner table with data in the organization table;
determining whether the data in the practitioner table matches the data in the organization table; and
creating a joins table that associates a practitioner with an organization.

20. The computer readable medium of claim 15, wherein the processing referral data includes:
grouping the referral data to identify sending and receiving providers;
grouping data by a filed to associate networks of sending and receiving providers;
grouping data by fields to associate networks of sending and receiving organization;
aggregating data by filter values and sum data for each filter value;
aggregating data by providers and summing referral counts for each sending and receiving provider; and
aggregating data by organizations and sum referral counts for sending and receiving organization.
